(19) United States
(12) Patent Application Publication
(10) Pub. No.: US 2018/0146886 A1
(43) Pub. Date: May 31, 2018

(54) Handpiece for Respiratory Monitor

(71) Applicant: Spirosure, Inc., Pleasanton, CA (US)

(72) Inventors: Ryan Leard, Oakland, CA (US); Greg Omweg, Livermore, CA (US); Brian Awabdy, Dublin, CA (US)

(73) Assignee: Spirosure, Inc., Pleasanton, CA (US)

(21) Appl. No.: 15/824,553

(22) Filed: Nov. 28, 2017

Related U.S. Application Data
(60) Provisional application No. 62/426,864, filed on Nov. 28, 2016.

(51) Int. Cl. A61B 5/087; A61B 5/097; A61B 5/091

(52) U.S. Cl. CPC A61B 5/087; G01N 33/497; A61B 5/091; A61B 5/097

(57) ABSTRACT

A device for use with a respiratory monitor is disclosed that includes features for collecting and guiding a breath gas sample from a user. The device includes a breath intake structure connected to a handheld body structure. The breath intake structure may include one or more permeable membranes to filter particular substances, such as bacteria, viruses, and microbes, from the breath gas sample before it proceeds through the body structure. The device further includes one or more pre-conditioning elements that pretreats the breath gas sample before it enters a respiratory monitor. In some embodiments, the device may include a mouthpiece and body that are used to collect and guide a breath gas sample to a nitric oxide detection device. Moisture is removed from the breath gas sample using a desiccant loaded into a pre-conditioning cartridge. The mouthpiece may be removably connected to the body and disposable. Interchangeable pre-conditioning cartridges may allow use of different cartridges and different pre-conditioning materials with the device.

### CROSS REFERENCE TO RELATED APPLICATIONS

This application claims the benefit of U.S. Provisional Application No. 62/426,864 filed on Nov. 28, 2016, the subject matter of which is herein incorporated by reference in its entirety.

### FIELD OF THE INVENTION

The invention relates to assemblies, component parts, and methods that are useful for respiratory diagnosis and monitoring; in particular, the invention relates to assemblies, component parts, and methods that are useful for use with breath-based diagnostic and monitoring devices.

### BACKGROUND OF THE INVENTION

Respiratory monitors have been used to detect biomarkers in a breath gas sample, such as a sample obtained from a patient's breath, and to diagnose and monitor respiratory conditions. Such devices may assist patients, clinicians, and other healthcare professionals in testing for particular constituents that are present in the breath. Accurate detection and measurement of certain breath constituents may help with the management of chronic pulmonary diseases such as asthma, Chronic Obstructive Pulmonary Disease (COPD), cystic fibrosis, and pulmonary hypertension.

Recent respiratory monitors include a wide range of diagnostic and monitoring technologies to test for a wide variety of substances. The devices are used in a wide variety of settings and for different medical applications. For example, fractional exhaled nitric oxide (FeNO) is a biomarker that indicates airway inflammation, which is a common side effect of patients with asthma and other pulmonary conditions. Point-of-care breath analyzers can provide FeNO information to a physician or in a clinical setting, while handheld or portable breath analyzers can provide exhaled nitric oxide information to an individual patient. Details regarding respiratory monitors useful for the detection of FeNO are described in U.S. Patent Publication No. 2015/0250408 A1, titled “Respiratory Monitor,” the entirety of which is incorporated by reference herein. Details regarding additional respiratory monitors useful for the detection of FeNO are described in U.S. Patent Publication No. 2017/0065208 A1, also titled “Respiratory Monitor,” the entirety of which is incorporated by reference herein. Respiratory devices using other sensors and other technologies also may test for various other biomarkers in a patient's breath.

Because respiratory monitors analyze breath gas samples obtained from a patient or other user, they require an effective way to collect the breath gas sample from the patient or user. They also require an effective way to guide the breath gas sample from a patient to the analyzer, while maintaining, to the extent possible, the original composition of the breath gas sample and minimizing the risks of outside contamination and leakage. These features may be particularly useful for respiratory monitors that employ highly sensitive gas component measuring sensors, in which the gas components are typically present in breath gas in minute amounts (e.g., parts per billion).

Further, certain respiratory monitors such as those employing nitric oxide (NO) sensors to determine the FeNO levels in a breath gas sample, need to provide accurate (NO) measurements in the presence of other gas components, including water and carbon dioxide (CO₂). At least some of these gas components may interfere with the ability of the respiratory monitor to accurately and efficiently test for particular biomarkers of interest. Thus, it may be desirable and advantageous to at least partially filter, remove, or screen for certain constituents in the breath gas sample before the sample enters the respiratory monitor for analysis.

In addition, in many instances it may be desirable to use a single respiratory monitor with multiple patients, to conduct multiple tests during different periods of time, and/or in multiple settings. As a result, it also may be advantageous to incorporate components that will allow for the convenient and economical use of the same respiratory monitor for different purposes, different users, and/or for different time periods, without cross-contaminating the breath gas samples and the equipment.

### BRIEF SUMMARY OF THE INVENTION

The present invention relates to one or more breath collection devices that are used in conjunction with one or more respiratory monitors. The device collects an exhaled breath gas sample from a user and guides the breath gas sample from the user through a flow pathway to a detection device or another breath-flow pathway. The device also may pre-condition the breath gas sample before it is analyzed in a detection device. Portions of the device may be configured to fit within a user's hand and include one or more removable components that may be inserted and then removed from the device to allow for periodic inspection, component replacement, component interchangeability, disposal as needed, and device maintenance.

In one embodiment, an assembly for use with a respiratory monitor comprises a breath intake section, an elongated body section, and a base section. The breath intake section comprises an opening dimensioned to accept a breath gas sample. The elongated body section comprises a pre-conditioning element adapted to remove one or more constituents from the breath gas sample. The base section comprises a base member in fluid communication with the body section and forming an outlet for a pre-conditioned breath gas sample.

In some embodiments, the base section may be adapted to be connected to a nitric oxide detection device, and the pre-conditioning element comprises a desiccant selected to at least partially remove moisture from the breath gas sample before it enters the nitric oxide detection device. The pre-conditioning element may comprise pre-conditioning media selected to at least partially remove one or more hydrocarbons from the breath gas sample. The elongated body section may further comprise a valve disposed upstream from the pre-conditioning element, and the valve is configured to permit the unidirectional flow of breath gas toward the pre-conditioning element. The breath intake section may further comprise a semi-permeable filter membrane fitted within the breath intake section. The breath intake section may be removably connected to the elongated body member. Further, the pre-conditioning element may comprise a removable cartridge.

In another embodiment, a handpiece assembly for use with a respiratory monitor comprises a mouthpiece, an elongated handle in fluid communication with the mouthpiece, and a base connector in fluid communication with the handle. The mouthpiece comprises an opening dimensioned to accept a breath gas sample. The elongated handle comprises material adapted to remove one or more constituents from the breath gas sample. The base connector forms an outlet for a pre-conditioned breath gas sample.

In addition, a method of pre-conditioning a breath gas sample for use with a respiratory monitor is described. The method comprises the steps of directing a breath gas sample through an opening of a breath intake member, flowing the breath gas sample from the breath intake member through a handheld body member in fluid communication with the breath intake member, flowing the breath gas sample in the handheld body member through a pre-conditioning material, reducing the amount of one or more constituents from the breath gas sample using the pre-conditioning material, and releasing the pre-conditioned breath gas sample to a respiratory monitoring device through a base member connected to the handheld body member.

In other embodiments, an assembly for use with a respiratory monitor comprises: a breath intake member, an elongated body member in fluid communication with the breath intake member, and an outlet member. The breath intake member includes an opening dimensioned to accept a breath gas sample from a user. The elongated body member includes a pre-conditioning member, and the outlet member includes a pathway through which pre-conditioned breath gas sample flows out of the assembly.

In some embodiments, the pre-conditioning member comprises a pre-conditioning cartridge. In a preferred embodiment, the pre-conditioning cartridge comprises a substantially cylindrical body, and the substantially cylindrical body comprises a length to diameter aspect ratio of approximately 2:1. In some instances, the pre-conditioning member comprises pre-conditioning media selected to at least partially remove one or more gas constituents from a breath gas sample before it enters a nitric oxide detection device. For example, the pre-conditioning media may comprise one or more desiccants selected to at least partially remove moisture from a breath gas sample. Alternatively, the pre-conditioning member may comprise pre-conditioning media selected to at least partially remove one or more hydrocarbons from a breath gas sample. In addition, the breath intake member further comprises a semi-permeable membrane through which a breath gas sample is filtered. The breath intake member may comprise, for instance, a semi-permeable membrane through which contaminants are at least partially filtered from a breath gas sample flowing therethrough. The breath intake member may be nested within an internal surface of an overlying breath intake member. The elongated body member also may comprise a valve configured to permit the unidirectional flow of breath gas to the pre-conditioning element. In a preferred embodiment, the valve reduces the intrusion of environment into the pre-conditioning element. The breath intake member may be removably connected to the elongated body member. The pre-conditioning member may be removably contained within the elongated body member. In addition, the opening of the breath intake member may comprise, without limitation, a substantially oval-shape.

In another embodiment, a handpiece assembly for use with a respiratory monitor includes a mouthpiece, an elongated handle in fluid communication with the mouthpiece, and a base connector. The mouthpiece includes an opening dimensioned to accept a breath gas sample from a user. The elongated handle is dimensioned to fit within a user's hand and includes a pre-conditioning element. The base connector is in fluid communication with the pre-conditioning element and includes a pathway through which pre-conditioned breath gas sample flows out of the handpiece assembly.

In yet another embodiment, the present invention provides a method of pre-conditioning a breath gas sample for use with a respiratory monitor. The method comprises the steps of: collecting a breath gas sample from an opening of a breath intake member and flowing the breath gas sample from the breath intake member through a handheld body member in fluid communication with the breath intake member. The breath gas sample is pre-conditioned as it flows through one or more pre-conditioning containers located in the body member. The pre-conditioned breath gas sample is released through an outlet member to a respiratory monitor.

### BRIEF DESCRIPTION OF THE DRAWINGS

Embodiments of the present invention may be described with reference to the accompanying drawings.

FIG. 1 illustrates schematically a perspective view of one embodiment of the handpiece of the present invention.

FIG. 2 illustrates schematically a side view of the internal components of a handpiece in accordance with one embodiment of the present invention. Here, one of the two casing sections has been removed for illustration purposes.

FIG. 3 illustrates schematically an exploded side view of the components of another embodiment of the present invention.

FIG. 4 illustrates schematically a partial perspective view of an upper portion of a handpiece in accordance with one embodiment of the present invention. The upper portion of the handpiece includes a mouthpiece fitted to a handle section that is partially shown in this figure.

FIG. 5 illustrates schematically a side view of a mouthpiece in accordance with one embodiment of the present invention.

FIG. 6 illustrates schematically an angled side view of a nozzle according to one embodiment of the present invention.

FIG. 7 illustrates schematically a side view of a pre-conditioning cartridge with pre-conditioning material in accordance with one embodiment of the present invention. The figure also illustrates schematically top and perspective views of connecting structures (O-rings and retainers) and adapters, respectively, used in this particular embodiment.

FIG. 8 illustrates schematically a perspective view of an end portion of a handpiece, in accordance with one embodiment of the present invention. In this embodiment, the end portion of the handpiece includes an adapter that connects to tubing.

### DETAILED DESCRIPTION OF THE INVENTION

As used herein, the terms “comprises,” “comprising,” “includes,” “including,” “has,” “having,” or any other variation thereof, are intended to cover a non-exclusive inclusion. For example, processes, methods, articles, or apparatuses that comprise a list of elements are not necessarily limited to only those elements but may include other elements not expressly listed or inherent to such processes, methods, articles, or apparatuses. Further, unless expressly stated to the contrary, “or” refers to an inclusive “or” but not to an exclusive “or.” For example, a condition A or B is satisfied by any one of the following: A is true (or present) and B is false (or not present), A is false (or not present) and B is true (or present), and both A and B are true (or present).

Also, use of “a” or “an” are employed to describe the elements and components of the invention. This is done merely for convenience and to give a general sense of the invention. This description includes one or at least one, and the singular also includes the plural unless it is obvious that it is meant otherwise.

Unless otherwise defined, technical and scientific terms used herein have the same meaning as commonly understood by one of ordinary skill in the art to which this invention belongs. Although methods that are similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, materials, methods, and examples are illustrative only and not intended to be limiting.

In the following description, numerous specific details, such as the identification of various system components, are provided to understand the embodiments of the invention. One skilled in the art will recognize, however, that embodiments of the invention can be practiced without one or more of the specific details, ordinary methods, components, materials, etc. In still other instances, well-known structures, materials, or operations are not shown or described in detail to avoid obscuring aspects of various embodiments of the invention.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or work characteristics may be combined in any suitable manner in one or more embodiments.

The present invention is directed generally to a device designed for use with a respiratory monitor. In some embodiments, an intake structure, such as a mouthpiece, collects a breath gas sample from a patient or other user. The intake structure guides the collected breath gas sample through a breath-flow pathway located in the body of the device, where the breath gas sample is treated and where one or more constituents is at least partially removed from the breath gas sample. The treated breath gas sample is then released from the device and directed to a respiratory monitor for testing and diagnostic purposes.

Among the potential advantages, the intake structures may comprise removable, replaceable, and/or disposable structures. The breath intake structures may be removable, replaceable, and/or disposable in their entirety. In other embodiments, the break intake structures may include portions or components that may be removable, replaceable, and/or disposable. In addition, the breath gas sample may be treated using material housed in removable cartridges, and different cartridges with different materials may be used interchangeably with the device. In a preferred embodiment, one or more membranes may be incorporated to filter particulate matter (e.g., bacteria, viruses, microbes, and/or other contaminants) from breath gas after it has entered the intake structure.

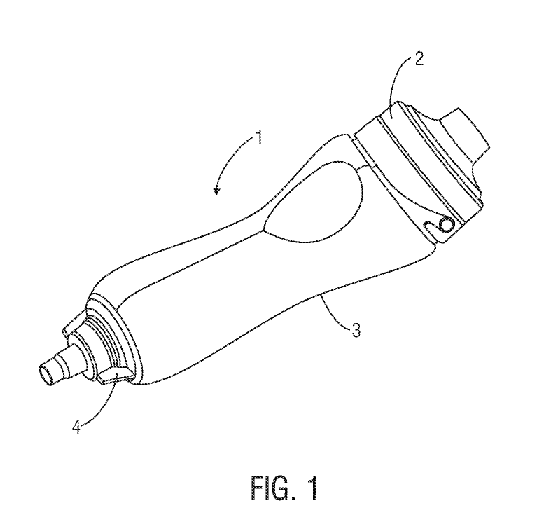

Referring to FIG. 1, an embodiment of the respiratory handheld device is shown. The device allows for the collection and pre-conditioning of a breath gas sample before it enters an analyzer for analysis. As an example, the device may selectively remove desired constituents (e.g., moisture, hydrocarbons, or other materials) from a breath gas sample before it is conveyed to a respiratory monitor for testing and analysis. The device 1 includes a mouthpiece 2 connected to an elongated handle section 3. The base 4 of the handle section may be connected via tubing (not shown) or other connecting structures to an analyzer (also not shown) for breath analysis.

When the device is being used, the device may be held in the hand, and a breath gas sample is introduced into the mouthpiece by exhaling into an inlet orifice in the mouthpiece. The breath gas sample is then guided through the handle section. In the handle section, the breath gas sample is pre-conditioned before it is routed to an analyzer for testing and/or another device for further treatment.

The mouthpiece illustrated is designed to direct a breath gas sample, for example, from a patient's mouth, through the inlet of a handheld device. The mouthpiece, and in particular, the inlet orifice of the mouthpiece, may be designed to facilitate the sealing of a user's lips around the edge of the mouthpiece. In this embodiment, the mouthpiece is a separate component that may be removably attached to the handle section through one or more connection structures known in the art to provide secure and, in preferred embodiments, substantially airtight connections for medical devices. Moreover, to avoid cross-contamination between multiple users or between different testing periods, the mouthpiece may be designed for use by a user and then discarded after one or more uses. The mouthpiece may be disposable, removed after it is used with the device, and replaced by another mouthpiece as needed, such as for different patients, different sampling periods, or different testing purposes. In a preferred embodiment, the mouthpiece may be stackable with other mouthpieces in their packaging. The stacking of one mouthpiece over another in this way reduces the overall volume required for shipping and storing multiple mouthpieces.

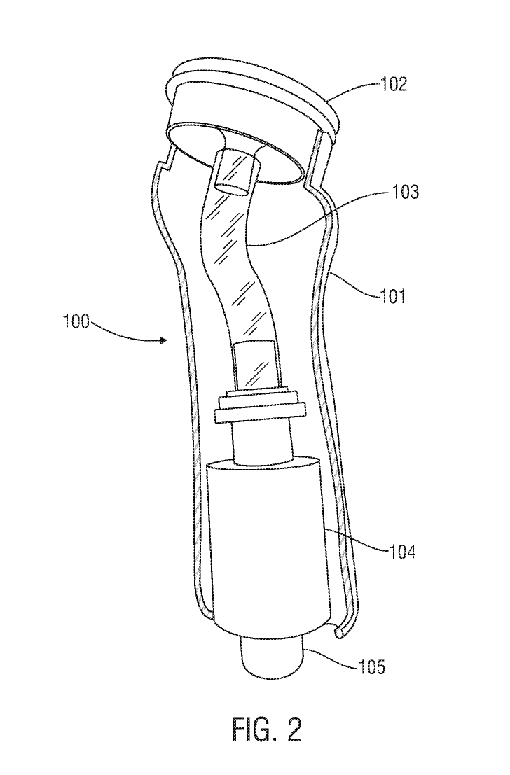

The internal components of one embodiment of the handle section are shown in FIG. 2. The handle section 100 includes an elongated handle 101 comprising two casing sections that may be separated and taken apart for internal access. For example, the casing sections of the handle may be taken apart for routine device maintenance, cleaning, or for the replacement of components within the device. In this figure, one of the two casing sections has been removed for illustration purposes.

Further referring to FIG. 2, a nozzle 102 receives a breath gas sample and directs the breath gas sample through a tube 103 to a substantially cylindrical pre-conditioning cartridge 104. The pre-conditioning cartridge may contain a desiccant (not shown) or other media known to selectively separate constituents from a breath gas sample. The base of the handle section includes a narrower portion 105 that fits around or otherwise connects to tubing or another gas conveying structure (not shown) to direct a treated breath gas sample to an analyzer for testing and/or a subsequent device for further treatment.

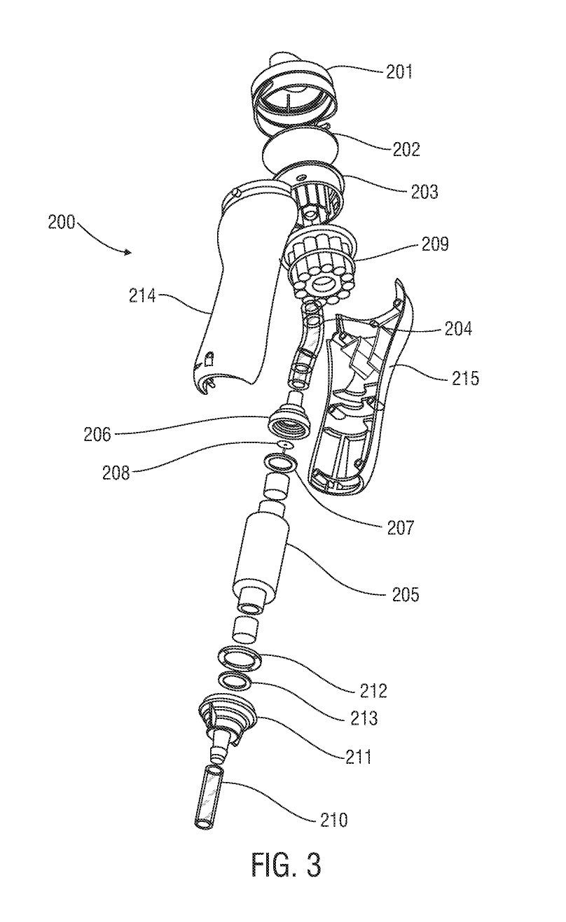

An exploded view of the components of another embodiment of the respiratory handheld device is shown in FIG. 3. The device 200 includes a mouthpiece 201 at one end of the device with a particle filter 202 inserted within the mouthpiece. In one embodiment, the particle filter may be formed from a paper sheet and cut into a shape that complements the shape of the mouthpiece area within which the filter is inserted. The filter acts as a particle screen (e.g., a bacterial filter, microbial filter, virus filter, and/or contaminant filter) to remove at least some of the unwanted particles before the breath gas sample proceeds through the breath-flow pathway. The filter may be made of materials known in the art to screen for desired particles, including, without limitation, paper comprising a polymer material, cotton material, or a cotton-polymer blend.

The particle filter used with the mouthpiece is permeable to certain breath gas constituents and not others. As such, the membrane may allow for the selective filtration of bacteria, microbes, viruses, particulates, and/or other substances from the breath gas sample. For added flexibility, different particle filters with different permeability and different membrane chemistry may be used with the mouthpiece, depending on the desired filtration characteristics. For example, particle filters with smaller sized openings may used to reduce the permeability of the particle filter to certain substances. Conversely, particle filters with larger sized openings may be used to allow increased flow through the device. As with the mouthpiece, the particle filter may be disposable, removed after it is used with the device, and replaced by another particle filter as needed, such as for different patients, different sampling periods, or different testing purposes.

Further referring to FIG. 3, a nozzle 203 is located adjacent to the mouthpiece and particle filter. The nozzle fits within the casing, between the casing sections of the handle section, and collects the breath gas sample introduced at the mouthpiece. The nozzle directs the breath gas sample through tubing 204 and into the gas flow path within the device. The nozzle provides a smooth surface, e.g., one with no or minimal seams, within which the breath may be substantially sealed to minimize leakage and guided from the mouthpiece into the breath path. In some embodiments, one or more optional components known to improve sealing within mechanical devices, including without limitation, one or more O-rings, may be included to further tighten the seal between the mouthpiece and the handle section. A pre-conditioning cartridge 205 may contain a desiccant, gel, or other material known to substantially remove constituents from a breath gas sample. An adapter 206 and O-ring 207 help to connect the tubing to the pre-conditioning cartridge and minimize or eliminate the leakage of the breath gas sample from the breath pathway.

As illustrated in FIG. 3, a one-way valve 208, also known as a “check valve,” may be inserted into the pre-conditioning assembly to facilitate the flow of a breath gas sample in one direction (e.g., downstream from the mouthpiece to the pre-conditioning cartridge). The check valve may prevent or reduce flow in the direction of the patient-user. In addition, the inclusion of one or more such valves may prevent or reduce the flow of ambient air into and out of the breath pre-conditioning cartridge (and downstream components) when the handpiece is not being used by a user-patient. In a preferred embodiment, the valve may be designed to be substantially impermeable to humidity. The inclusion of such a one-way valve in the pre-conditioning assembly reduces the amount of humidity that is allowed to move hydroscopically through the breath-flow pathway, thus minimizing the amount of moisture present in the breath gas sample flowing through the pre-conditioning cartridge and extending the usable life of the media contained within the pre-conditioning cartridge.

Again referring to FIG. 3, one or more metal dowels 209 may be optionally included within the device to provide weight and added structural stability. In this embodiment, a bundled set of steel rods is used for weight and added stability. In practice, however, any material in any combination of known sizes, shapes, and configurations that are known in the art to provide weight and added stability may be used.

The base of the pre-conditioning cartridge is connected to tubing 210 using a breath tube connector 211 fitted with a retainer 212 and O-ring 213. The internal components of the handle section are enclosed within handle casing sections 214 and 215. As shown, the handle casing sections may include internal protrusions, notches, and grooves to help support and secure the components within the casing. A barb fitting, located along the base of the breath tube connector, extends below the base of the handle sections to facilitate a substantially airtight connection of the handpiece to the tubing.

When in use, a breath gas sample may be introduced through the mouthpiece, e.g., from a user exhaling into the mouthpiece. The breath gas sample is directed from the nozzle into the pre-conditioning cartridge containing pre-conditioning material. The breath gas sample is known to contain multiple constituents, some of which may affect the effectiveness of breath analysis. The pre-conditioning cartridge may be loaded with one or more pre-conditioning materials to improve analysis results by substantially removing one or more constituents of interest. For example, desiccants may be used to remove moisture from a breath gas sample before it is directed to a nitric oxide monitor for testing. Other materials may be used to remove other substances from a breath gas sample (e.g., hydrocarbons). The treated breath gas sample is then directed through external tubing to a separate analyzer for analysis and/or subsequent treatment.

A single pre-conditioning cartridge is shown in his embodiment; however, it may be appreciated that multiple types of pre-conditioning cartridges with a variety of conditioning materials may be used in various combinations to selectively treat incoming breath gas samples. For example, a pre-conditioning cartridge may be removed after use and/or after the useful life of the pre-conditioning material contained in the cartridge, and the pre-conditioning cartridge may be replaced with a new cartridge. Also, the device may be used with interchangeable pre-conditioning cartridges containing customized media suitable for different uses.

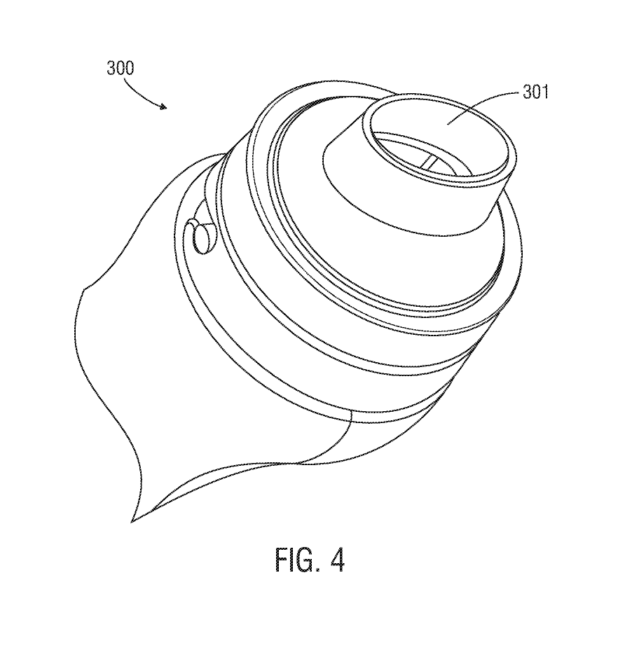

The handheld respiratory device of the present invention may be designed for use with a wide variety of mouthpiece shapes, sizes, designs, and dimensions to accommodate different uses and breath maneuvers. An example of a mouthpiece used with the handheld respiratory device is illustrated in FIG. 4. In this example, an ovalized mouthpiece 300 has a substantially oval-shaped orifice 301. The orifice may be sized to allow for use by a wide variety of patients. In a preferred embodiment, the raised oval-shaped ring that forms the orifice and around which a user's lips may fit may have a draft angle of approximately eight degrees.

The shapes, orientations, and designs of the mouthpiece, orifice, and the raised mouthpiece ring, however, are provided here for illustration purposes. Additional orifice and ring shapes, orientations, and designs are also within the contemplated scope of this invention. For example, the mouthpiece orifice and the corresponding ring that forms the mouthpiece orifice may be substantially circular or rectangular in shape. It may include curved, straight, or a combination of curved and straight edges or grooves. The mouthpiece may include radial and other protrusions that extend from the bottom of the mouthpiece at different angles.

In this embodiment, the edges of the orifice may allow for the creation of a seal around the mouthpiece and fit around the lips of a user. In a further preferred embodiment, the mouthpiece may be designed to receive an exhaled breath gas sample with minimal effort by the user to fit his or her lips around the edges of the mouthpiece. The mouthpiece also may be designed to receive an exhaled breath gas sample with minimal leakage of the breath gas sample to the surrounding air.

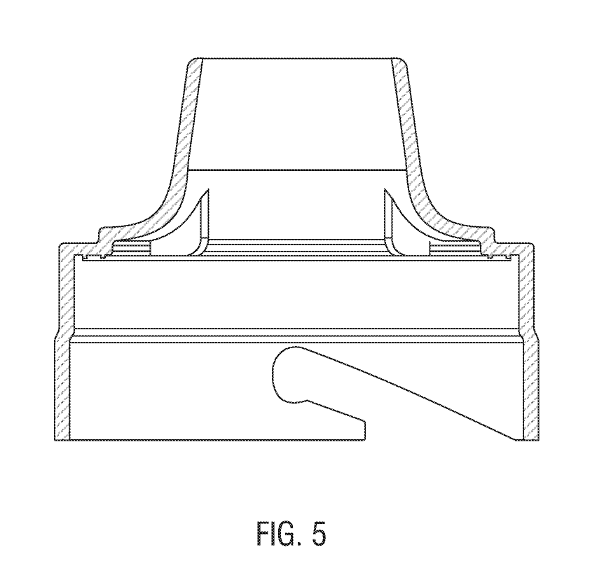

The mouthpiece connects to the handle section in one or more ways known in the art to securely connect mechanical components. For example, as shown in FIG. 5, in this embodiment, the mouthpiece has one or more notches that allows the mouthpiece to interlock with one or more complementary pegs located on the handle section, e.g., by twisting the mouthpiece against guiding protrusions located on the inside of the mouthpiece. The guiding protrusions are designed to fit along complementary groves of the handle section. A user may twist the mouthpiece until the notch of the mouthpiece meets the complementary peg on the handle section. In a preferred embodiment, the mouthpiece removably interlocks with the handle section to allow for a secure connection, interchangeability, removal, and replacement of the mouthpiece as desired for different users and multiple uses.

It will be appreciated that the described mechanism for connecting the mouthpiece to the handle section is not limiting. For example, the mouthpiece may be connected through the use of other structures known in the art. They include without limitation the use of one or more (or a combination of): mechanical connections, interconnecting parts, locks, latches, complementary grooves and notches, protrusions, pegs, adhesives, glues, pins, snaps, snap-in connectors, twist-on fittings, hooks, screws, mechanical fittings, adapters, retainers, O-rings, or other connecting structures and connecting materials.

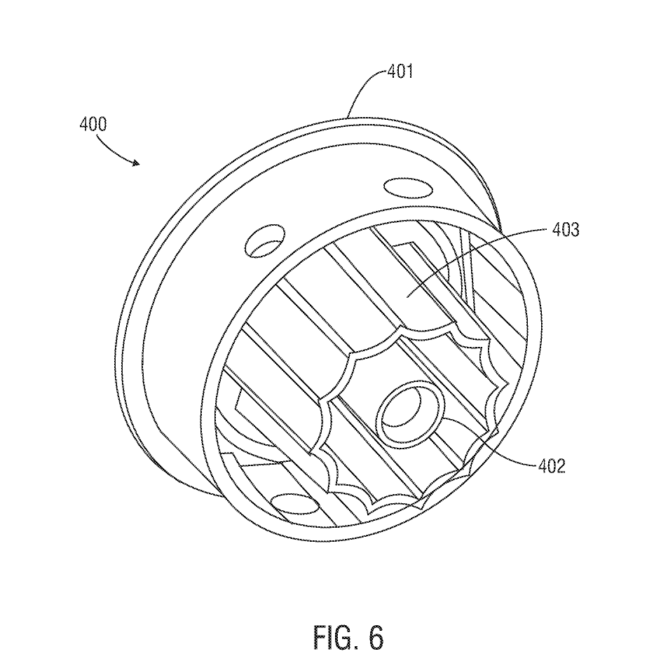

As discussed, an incoming breath gas sample enters the mouthpiece and flows through the nozzle, which helps to direct the breath gas sample into the fluid path for the device. A nozzle of one embodiment of the present invention is shown in FIG. 6. The breath gas sample may enter the nozzle 400 through an opening 401 located along the top portion of the nozzle. In a preferred embodiment, the inside of the nozzle may form a funnel-like surface that tapers from the wider opening along the top portion toward a narrower cannula 402 along a bottom portion of the nozzle. Following this funnel-like surface, the breath flow is directed from the space formed by the wider opening along to the top portion of the nozzle until it reaches the space formed by the narrower cannula. An end of the cannula may be connected to tubing to allow for flow of the breath gas sample through the device. In a preferred embodiment, the nozzle may also include a plurality of recessed ridges 403 located along one or more internal surfaces, against or near which one or more metal dowels (as shown in FIG. 3) may rest.

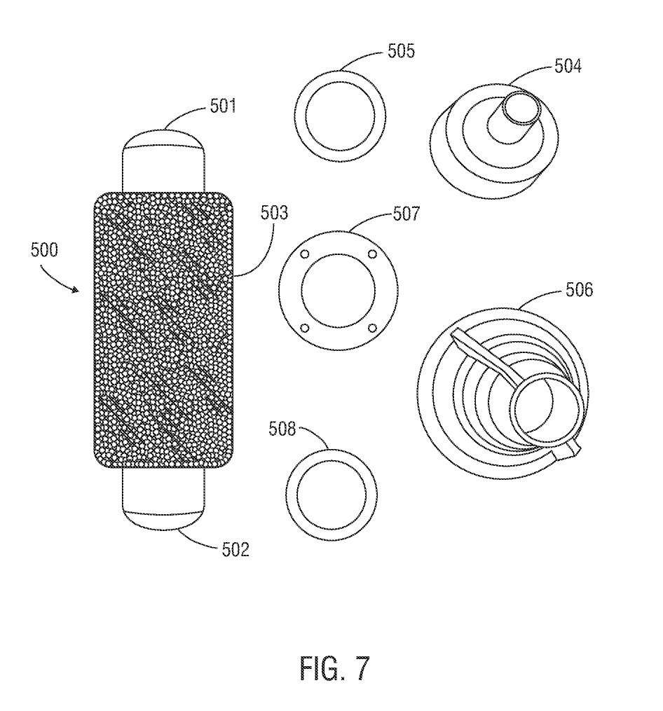

As described in this invention, the handheld respiratory device may be used with one or more pre-conditioning materials to treat a breath gas sample. As a non-limiting example, the handheld respiratory device may remove one or more constituents from the breath gas sample before it is conveyed to a respiratory monitor for testing and diagnosis. The pre-conditioning material may be housed in one or more cartridges, which may include, without limitation, a substantially cylindrically-shaped body. An exemplary pre-conditioning cartridge and associated components are illustrated in FIG. 7. A substantially cylindrical pre-conditioning cartridge includes a substantially cylindrical main body 500 and ends 501 and 502. Each end has a reduced diameter compared to the main body. Foam or other materials may be inserted into the rods. The cartridge may house one (or more) of various pre-conditioning materials known in the art to be effective in removing one or more desired substances of interest from a breath gas sample before it enters an analyzer for testing. The shape, orientation, and design of the cartridges is described here for illustration purposes, however, and pre-conditioning cartridges comprising other shapes, sizes, and aspect ratios also may be used.

Again referring to the example of FIG. 7, in one embodiment, the pre-conditioning material may include one or more desiccants 503 to help remove moisture from a breath gas sample before it is introduced to a nitric oxide monitor for analysis and diagnosis. Removing humidity and lowering the moisture content in the breath gas sample may help to improve the results of the subsequent breath analysis. As such, a humidity sensor may be fitted to monitor the humidity of a breath gas sample before it flows to an analyzer for testing. Suitable desiccants include, without limitation: zeolites, gels, calcites, calcium-based materials, silica gels, and other substances known in the art to be effective in removing moisture from a gas sample. In a preferred embodiment of the present invention, the main body of the pre-conditioner has a length to diameter aspect ratio of approximately 2:1. This ratio aids the pre-conditioning media (e.g., a desiccant) in effectively removing moisture for a range of typical flow rates for breath gas. The media may thus be used for a longer period of time. For example, this aspect ratio may allow for the effective removal of moisture using a 3A zeolite when flowing 85% relative humidity (RH) air at up to approximately 3.5 liters per minute, +/−10%.

In at least some instances, it may be desirable to treat the pre-conditioning material before it is used in the handpiece to improve the performance of the pre-conditioning material. For example, during nitric oxide testing of a breath gas sample, contacting the breath gas sample with an untreated pre-conditioning material (e.g., type 3A zeolite) may affect the nitric oxide concentration present in the sample. As such, in a preferred embodiment, the pre-conditioning material may be treated to optimize the nitric oxide concentration before it is used in the conditioning cartridges to minimize any detrimental affects on the nitric oxide levels.

Other pre-conditioning materials may be used to remove other constituents from a breath gas sample. For example, pre-conditioning materials may be selected to remove hydrocarbons from a breath gas sample. In some embodiments, the pre-conditioning cartridges may be designed to be removable to allow for refill, the use of different pre-conditioning materials, the replacement of cartridges, and maintenance. The pre-conditioning cartridges may also be disposable and may be removed from the device; e.g., after the pre-conditioning material contained within the cartridge is no longer useful or needed for its intended purpose. In some embodiments, the pre-conditioning cartridges may be refillable. For example, pre-conditioning material within the cartridges may be refilled after the useful life of the material. The pre-conditioning cartridges may be removably connected within the handpiece to allow for insertion, removal, replacement, refill and/or changing of the cartridges, as needed. In a preferred embodiment, the pre-conditioning cartridge is formed from glass; however, other materials that are known in the art to securely house preconditioning substances (e.g., plastic, rubber, aluminum, metals, or ceramic materials) may also be used.

The pre-conditioning cartridge may be incorporated into the handle section in one of many ways known in the art. Again referring to FIG. 7, in this embodiment, an upper adapter 504 is fitted over the upper rod 501 of the cartridge on one end and with tubing (not shown) within the handle section on the other end. An O-ring 505 helps to secure the connection along the upper portion of the cartridge. A lower adapter 506 is fitted over the lower rod 502 of the cartridge on one end and with tubing (not shown) extending from the handle section on the other end. A retainer 507 and O-ring 508 help to secure the connection along the lower portion of the cartridge.

It will be appreciated that the described mechanisms for incorporating the pre-conditioning cartridge into the handpiece is not limiting. The pre-conditioning cartridge of the present invention may be incorporated through the use of other structures and materials known in the art. They include without limitation the use of one or more (or a combination of): mechanical connections, interconnecting parts, locks, latches, complementary grooves and notches, twist-on structures, protrusions, pegs, adhesives, glues, pins, snaps, snap-in connectors, twist-on fittings, hooks, screws, fittings, adapters, retainers, O-rings, or other connecting structures and materials.

As discussed above, a one-way valve may be incorporated to restrict the flow of the breath gas sample in one direction, thereby limiting the amount of humidity that enters the pre-conditioning cartridge and extending the life of the pre-conditioning material (e.g., desiccant or other material). In a preferred embodiment, the check valve (not shown) is adjacent to the entrance of the cartridge and rests within the upper adapter.

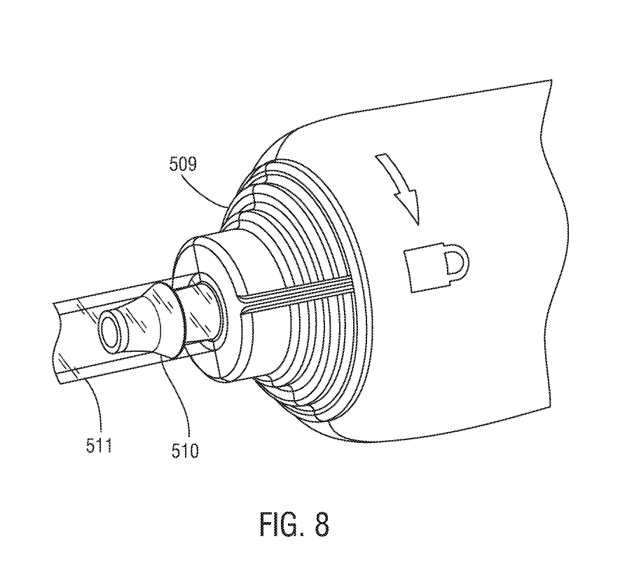

The base of the device may be connected to an external respiratory analyzer through a breath tube connector and tubing. In some embodiments, as shown in FIG. 8, the lower cartridge adapter 509 extends past the handpiece casing and includes an elongated barb fitting 510. Tubing 511 may be inserted over the barb fitting to connect the device to an analyzer.

The handpiece of the present invention can be adapted for use with a wide variety respiratory monitoring devices. They may be used with respiratory monitors that are used to measure different biomarkers in a breath gas sample, for example. The handpiece of the present invention also may be used to precondition breath gas samples before they are transmitted to respiratory monitors that use a wide variety of different technologies to analyze breath gas samples with varying sensitivities.

Although the dimensions and shapes of the handpiece and the components used, and the sizes of the openings are described here in particular embodiments, the ordinary artisan will recognize that the dimensions of the handpiece and the components used, and the sizes of the openings in the present invention may vary to accommodate different design considerations, such as without limitation: desired breath-flow pathways, different users, desired overall weight of the handpiece, desired volume available for breath flow, different breath flow rates, ease of fabrication, material selection, desired capacity, breath flow volume, desired contact times, and/or the desired access space for inspection, component replacement, and maintenance purposes.

The layout of the handpiece structures, components, and members described above may be designed in a variety ways and may depend on such factors such as, without limitation: desired breath-flow pathways, different users, desired overall weight of the handpiece, desired volume available for breath flow, different breath flow rates, ease of fabrication, material selection, desired capacity, breath flow volume, desired contact times, and/or the desired access space for inspection, component replacement, and maintenance purposes. In some embodiments, for example, one or more of the handpiece structures or sections may be integrated to form a unitary piece for ease of construction.

The ordinary artisan will also recognize that the absolute dimensions of the openings can be selected to accept industry standard connections/fittings (e.g., tubing, pipes, or other gas conveying structures). As described above, the openings for breath flow are shown as substantially curved or rounded. However, any of a multitude of complementary shapes allowing gas flow through the handpiece and well known to the ordinary artisan could be used. Additional structures, including without limitation additional adapters, fittings, and connectors, may be used to improve the connections and facilitate breath flow through the device. Such fittings can offer flexible and substantially airtight connections between the handpiece components and connections for breath flow into and out of the handpiece.

The ordinary artisan can also recognize that other materials commonly used in applications involving medical device applications can be employed in the present invention. In a preferred embodiment, and unless otherwise described in this application, plastic materials may be used due to the greater ease of pre-molding such structures. But generally any material that can be molded or cast might be used to fabricate the handpiece components, including but not limited to polypropylene, high-density polyethylene (HDPE), low-density polyethylene (LDPE), rubber, aluminum, and other suitable metals.

The above disclosure is sufficient to enable one of ordinary skill in the art to practice the invention, and provides the best mode of practicing the invention presently contemplated by the inventor. While there is provided herein a full and complete disclosure of specific embodiments of this invention, it is not desired to limit the invention to the exact construction, dimensional relationships, and operation shown and described. Various modifications, alternative constructions, design options, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

1-42. (canceled)

43. An assembly for use with a respiratory monitor, comprising:
a. a breath intake section comprising an opening dimensioned to accept a breath gas sample;
b. an elongated body section in fluid communication with the breath intake section, the elongated body section comprising a pre-conditioning element adapted to remove one or more constituents from the breath gas sample; and
c. a base section in fluid communication with the elongated body section, the base section forming an outlet for a pre-conditioned breath gas sample.

44. The assembly of claim 43, wherein the base section is adapted to be connected to a nitric oxide detection device, and the pre-conditioning element comprises a desiccant selected to at least partially remove moisture from the breath gas sample before it enters the nitric oxide detection device.

45. The assembly of claim 43, wherein the pre-conditioning element comprises pre-conditioning media selected to at least partially remove one or more hydrocarbons from the breath gas sample.

46. The assembly of claim 43, wherein the elongated body section further comprises a valve disposed upstream from the pre-conditioning element, and further wherein the valve is configured to permit the unidirectional flow of breath gas toward the pre-conditioning element.

47. The assembly of claim 46, wherein the valve comprises a structure that is substantially impermeable to humidity.

48. The assembly of claim 43, wherein the breath intake section further comprises a semi-permeable filter membrane within the breath intake section.

49. The assembly of claim 43, wherein the breath intake section is removably connected to the elongated body section.

50. The assembly of claim 43, wherein the pre-conditioning element comprises a removable cartridge.

51. A handpiece assembly for use with a respiratory monitor comprising:
a. a mouthpiece comprising an opening dimensioned to accept a breath gas sample;
b. an elongated handle in fluid communication with the mouthpiece, the handle comprising material adapted to remove one or more constituents from the breath gas sample; and
c. a base connector in fluid communication with the handle and forming an outlet for a pre-conditioned breath gas sample.

52. The handpiece assembly of claim 51, wherein the base connector is adapted to release the pre-conditioned breath gas sample to a nitric oxide detection device, and the material comprises a desiccant selected to at least partially remove moisture from the breath gas sample before it enters the nitric oxide detection device.

53. The handpiece assembly of claim 51, wherein the material comprises a material selected to at least partially remove one or more hydrocarbons from a breath gas sample.

54. The handpiece assembly of claim 51, wherein the elongated handle further comprises a valve disposed upstream from the material, and further wherein the valve is configured to permit the unidirectional flow of breath gas toward the material.

55. The handpiece assembly of claim 51, wherein the elongated handle further comprises a valve disposed upstream from the material, and further wherein the valve comprises a structure that is substantially impermeable to humidity.

56. The handpiece assembly of claim 51, further comprising a semi-permeable filter membrane adjacent to the mouthpiece.

57. The handpiece assembly of claim 51, wherein the mouthpiece is removably connected to the handle.

58. The handpiece assembly of claim 51, wherein the material is housed within a removable cartridge.

59. A method of pre-conditioning a breath gas sample for use with a respiratory monitor, comprising:
a. directing a breath gas sample through an opening of a breath intake member;
b. flowing the breath gas sample from the breath intake member through a handheld body member in fluid communication with the breath intake member;
c. flowing the breath gas sample in the handheld body member through a pre-conditioning material;
d. reducing the amount of one or more constituents from the breath gas sample using the pre-conditioning material; and
e. releasing the pre-conditioned breath gas sample to a respiratory monitoring device through a base member connected to the handheld body member.

60. The method of claim 59, wherein the respiratory monitoring device comprises a nitric oxide detection device, and the pre-conditioning material comprises a desiccant selected to at least partially remove moisture from the breath gas sample before it enters the nitric oxide detection device.

61. The method of claim 59, the step of (d) reducing the amount of one or more constituents from the breath gas sample using the pre-conditioning material comprises removing one or more hydrocarbons from the breath gas sample.

62. The method of claim 59, wherein the handheld body member further comprises a valve disposed upstream from the pre-conditioning material, and the valve is configured to permit the unidirectional flow of breath gas toward the pre-conditioning material.

63. The method of claim 62, wherein the valve comprises a structure that is substantially impermeable to humidity.

64. The method of claim 59, further comprising the step of directing the breath gas sample entering the breath intake member through a semi-permeable filter membrane fitted within the breath intake member.

64. The method of claim 59, wherein the breath intake member is removably connected to the handheld body member.

66. The method of claim 59, wherein the pre-conditioning material is housed within a removable cartridge.
